(19) United States
(12) Patent Application Publication
(10) Pub. No.: US 2025/0331894 A1
(43) Pub. Date: Oct. 30, 2025

(54) PREPARATION METHOD FOR TEMPERATURE-SENSITIVE SUBMUCOSAL INJECTION FOR ENDOSCOPIC SUBMUCOSAL DISSECTION

(71) Applicant: SHENGJING HOSPITAL OF CHINA MEDICAL UNIVERSITY, SHENYANG (CN)

(72) Inventors: Siyu SUN, Shenyang (CN); Yang LIU, Shenyang (CN); Guangwei SUN, Shenyang (CN)

(21) Appl. No.: 19/263,486

(22) Filed: Jul. 9, 2025

Related U.S. Application Data
(63) Continuation of application No. PCT/CN2023/143140, filed on Dec. 29, 2023.

(30) Foreign Application Priority Data
Feb. 16, 2023 (CN) 202310122338.3

(51) Int. Cl. A61B 17/34; A61K 31/722

(52) U.S. Cl. CPC A61B 17/3478; A61K 31/722

(57) ABSTRACT

A method for preparing a thermosensitive submucosal injection for endoscopic submucosal dissection (ESD), relating to the technical field of regenerative medicine. The specific method involves constructing a novel thermosensitive submucosal injection solution capable of sustained-release hemostatic drugs and growth factors using high-pH chitosan, polyvinylpyrrolidone, calcium ions, and disodium β-glycerophosphate. This injection has very low viscosity, does not require special injection devices, is able to be injected using conventional endoscopic injection needles currently used in clinical practice, and exhibits high injection rate. Additionally, after being injected into the submucosal layer, this injection rapidly forms a high-strength hydrogel under body temperature, more stably supporting the raised state of the mucosal layer to facilitate ESD operations. It also reduces intraoperative and postoperative bleeding, promotes postoperative wound healing, and holds significant clinical practical application value.

### CROSS REFERENCE TO RELATED APPLICATION

This application is a continuation of International Patent Application No. PCT/CN2023/143140 with a filing date of Dec. 29, 2023, designating the United States, now pending, and further claim priority of Chinese application 202310122338.3, filed Feb. 16, 2023. The content of the aforementioned applications, including any intervening amendments thereto, are incorporated herein by reference.

### TECHNICAL FIELD

The present disclosure relates to the technical field of regenerative medicine, and in particular to a method for preparing a thermosensitive submucosal injection for endoscopic submucosal dissection and a use thereof.

### BACKGROUND

Early-stage gastrointestinal lesions are primarily located in the mucosa and submucosa. Since endoscopic submucosal dissection (hereinafter referred to ESD) may remove relative large lesions, achieves a complete resection rate of over 90%, and may obtain pathological diagnostic samples, ESD has thus become the main diagnostic and therapeutic approach for early-stage gastrointestinal lesions.

In recent years, although ESD has achieved significant advancements, the prolonged procedure time and high technical difficulty in cutting and dissecting the mucosa result in a persistently high probability of complications such as perforation and bleeding during surgery, even when performed by experienced endoscopists, which limits the widespread adoption of ESD.

To reduce complications, submucosal injections are typically injected beneath the mucosal layer of lesions to cause mucosal layer elevation, thereby lifting the lesioned mucosa and separating it from the muscular layer. This provides operators with better visual fields and operating space to minimize vascular damage and avoid injury to the muscular layer. Consequently, submucosal injections are critical for enhancing the safety and efficacy of ESD therapy.

Currently, clinically common submucosal injections include normal saline, hypertonic saline, hypertonic glucose solutions, glycerol fructose injection, and sodium hyaluronate containing medical colorants such as methylene blue. Normal saline is the most commonly used submucosal injection. However, being isotonic, it is rapidly absorbed by surrounding tissues after submucosal injection, causing a significant decrease in mucosal layer elevation height within 5 minutes, which necessitates repeated injections and increases procedural complexity. Although hypertonic saline, glucose solutions, and glycerol fructose injection show improved efficacy compared to normal saline, the maintenance of mucosal elevation remains suboptimal, with elevation height reducing to below 50% within 20 minutes. Moreover, their hypertonicity causes mucosal damage and may induce wound ulcers. Hyaluronic acid is expensive and carries the risk of stimulating residual tumor tissue growth, making it unsuitable for widespread use. Therefore, current clinically used submucosal injections fail to meet ESD requirements, and there is an urgent need to develop new submucosal injections to overcome the limitations of the prior art, stably maintain mucosal layer elevation, and facilitate ESD operations.

### SUMMARY

To address the aforementioned issues, provided in the present disclosure is a method for preparing a thermosensitive submucosal injection for endoscopic submucosal dissection and a use thereof, aiming to stably maintain mucosal layer elevation to facilitate ESD operations, reduce intraoperative and postoperative bleeding, and promote postoperative wound healing.

To achieve the above objectives, the following technical solutions are provided by the present disclosure.

Provided in the present disclosure is a method for preparing a thermosensitive submucosal injection for endoscopic submucosal dissection. The method includes the following steps:
S1. adding a polyvinylpyrrolidone solution to a chitosan solution with a high-pH value under ice bath and stirring conditions, followed by stirring and mixing uniformly to prepare a mixed solution A;
S2. adding a disodium β-glycerophosphate solution dropwise to the mixed solution A of S1, followed by stirring and mixing uniformly to prepare a mixed solution B;
S3. adding a calcium ion solution to the mixed solution B prepared in S2, followed by stirring and mixing uniformly to prepare a mixed solution C;
S4. adding a medical color indicator to the mixed solution C prepared in S3, followed by stirring and mixing uniformly to prepare a mixed solution D;
S5. adding a hemostatic drug epinephrine to the mixed solution D prepared in S4, followed by stirring and mixing uniformly to prepare a mixed solution E;
S6. adding a growth factor to the mixed solution E prepared in S5, followed by stirring and mixing uniformly to obtain the thermosensitive submucosal injection solution.

In some implementations, the high-pH value in step S1 is in a range of 5.5 to 6.2.

In some implementations, in step S1, the chitosan has a degree of deacetylation of 95% or more and a molecular weight of 5,000-300,000 Da; the polyvinylpyrrolidone has a molecular weight of 20,000-300,000 Da.

In some implementations, specific conditions of the stirring in steps S1 to S6 are: a stirring and mixing time of 20 to 40 minutes in step S1; a stirring and mixing time of 10 to 20 minutes in steps S2 to S6; and a stirring speed of 1,000 to 10,000 rpm in steps S1 to S6.

In some implementations, the calcium ion solution in step S3 includes one or more of calcium chloride, calcium heparin, calcium lactate, and calcium gluconate.

In some implementations, the medical color indicator in step S4 includes any one of methylene blue and indigo carmine, or a combination thereof.

In some implementations, the growth factor in step S6 includes any one of bFGF, VEGF, EGF, PDGF, TGF-0, and HGF, or a combination thereof.

In some implementations, in the thermosensitive submucosal injection solution prepared in step S6: the chitosan has a concentration of 0.3 to 1% (w/v, g/mL); the polyvinylpyrrolidone has a concentration of 2 to 10% (w/v, g/mL); the disodium β-glycerophosphate has a concentration of 2 to 10% (w/v, g/mL); the calcium ions have a concentration of 0.01 to 0.05% (w/v, g/mL); the medical color indicator has a concentration of 1% (v/v); the epinephrine has a concentration of 0.005 to 0.02% (v/v); and each growth factor has a concentration of 20 to 100 ng/mL.

Provided in the present disclosure is further a use of the thermosensitive submucosal injection prepared by any one of the aforementioned method, which includes injecting the thermosensitive submucosal injection locally into a submucosal layer through an endoscopic injection needle.

In some implementations, each injection site receives 0.5 to 1.5 mL of the thermosensitive submucosal injection, and the injection is performed at a rate of 5 to 15 mL/min.

Compared to the prior art, the present disclosure provides beneficial effects as follows.
1. The present disclosure employs high-pH chitosan, polyvinylpyrrolidone, calcium ions, and disodium β-glycerophosphate to construct a novel three-dimensional molecular-coupled thermosensitive gel sustained-release system. Specifically, based on the traditional chitosan/disodium β-glycerophosphate system, high-pH chitosan is utilized to improve injectability and shorten gelation time, while polyvinylpyrrolidone and calcium ions further crosslink chitosan through hydrogen bonds and coordination bonds, synergistically enhancing the stability, integrity, and strength of the hydrogel. This enables the injection solution to rapidly form a more complete and stronger hydrogel after being injected into the submucosal layer, thereby more stably supporting the raised state of the mucosal layer to facilitate ESD operations, while also enhancing the sustained-release capacity for hemostatic drugs and growth factors.
2. The injection of the present disclosure employs a high-pH chitosan solution (pH range of 5.5 to 6.2). Compared with conventional low-pH solutions (pH≤5.4), the high-pH chitosan solution exhibits significantly enhanced electrostatic attraction between chitosan molecules. When disodium β-glycerophosphate is added, its thermosensitivity is markedly improved, with gelation time significantly shortened. Additionally, to achieve injectability through endoscopic injection needles, the injection requires low-concentration chitosan solutions. The high-pH chitosan solution maintains rapid gelation capability even after dilution at low concentrations, with low viscosity and excellent injectability suitable for endoscopic applications, whereas conventional low-pH chitosan solutions fail to form gels. Furthermore, chitosan, the only cationic polysaccharide in nature, possesses superior antibacterial properties, film-forming characteristics, and wound-healing promotion capabilities. After ESD resection, the wound remains covered by the thermosensitive gel layer, which forms an antibacterial film to protect the wound surface, thereby providing critical antibacterial effects and promoting wound healing.
3. In addition to rapid gelation, gel strength is also a critical factor in maintaining mucosal layer elevation. The thermosensitive submucosal injection proposed in the present disclosure employs polyvinylpyrrolidone and calcium ions to synergistically enhance the strength of the chitosan-based thermosensitive gel, thereby providing better support for mucosal layer elevation. Polyvinylpyrrolidone forms strong hydrogen bonding interactions with chitosan molecules, creating a three-dimensional interpenetrating network that further reinforces the gel network strength. Additionally, this molecular interpenetration effectively shields the electrostatic attraction between chitosan molecules, ensuring greater stability of the thermosensitive gel at low temperatures. Calcium ions engage in coordination interactions with both chitosan and polyvinylpyrrolidone. The addition of calcium ions further crosslinks these two polymers, increases the structural ordering of the three-dimensional network, and enhances gel strength, thereby effectively slowing the collapse of the elevated mucosal layer.
4. The injection proposed in the present disclosure contains the hemostatic drug epinephrine, which may reduce intraoperative bleeding during ESD procedures to maintain clear visual fields and shorten surgical time. Additionally, the thermosensitive gel layer remaining on postoperative wounds enables sustained-release of epinephrine, effectively mitigating postoperative bleeding complications.
5. The thermosensitive submucosal injection of the present disclosure further contains multiple wound-healing-promoting growth factors. The retained thermosensitive gel layer on postoperative wounds allows sustained-release of these growth factors to accelerate wound healing and reduce scarring.
6. The thermosensitive submucosal injection proposed in the present disclosure exhibits very low viscosity, requires no special injection devices, and may be administered using conventional endoscopic injection needles currently employed in clinical practice. It demonstrates high injection rate, rapid mucosal elevation, fast gelation, slow collapse of elevated mucosa, and eliminates the need for repeated injections. These characteristics fully meet the specific requirements of ESD procedures, significantly reduce surgical difficulty, markedly shorten operation time, and alleviate complications.

### BRIEF DESCRIPTION OF THE DRAWINGS

FIG. 1 shows the effects of different polyvinylpyrrolidone and calcium ion concentrations on mucosal layer collapse. Inset A represents the experimental group; inset B represents control group 1; and inset C represents control group 2.

### DETAILED DESCRIPTION

To provide clearer understanding of the objectives, technical solutions, and advantages of the present disclosure, the following embodiments are described in detail with reference to specific examples. It should be understood that the specific embodiments described herein are merely illustrative of the present disclosure and shall not be construed as limiting the scope thereof.

The present disclosure constructs a thermosensitive submucosal injection capable of sustained-release hemostatic drugs and growth factors using high-pH chitosan, polyvinylpyrrolidone, calcium ions, and disodium β-glycerophosphate. After being injected into the submucosal layer, this injection rapidly forms a high-strength hydrogel under body temperature, more stably supporting the raised state of the mucosal layer to facilitate ESD operations, while reducing intraoperative and postoperative bleeding and promoting postoperative wound healing.

### Example 1

Under ice bath and stirring conditions (3,000 rpm), a polyvinylpyrrolidone solution (molecular weight: 100,000 Da) was first added to a chitosan solution (degree of deacetylation: 98%, molecular weight: 100,000 Da, pH 6.1), followed by stirring and mixing for 25 minutes. Subsequently, a disodium β-glycerophosphate solution was dropwise added to the above mixture, followed by stirring and mixing for 13 minutes. Calcium heparin solution was then added, followed by stirring and mixing for 16 minutes. Next, the medical color indicator methylene blue was added, followed by stirring and mixing for 12 minutes. The hemostatic drug epinephrine was subsequently added, followed by stirring and mixing for 17 minutes. Finally, growth factors bFGF, PDGF, and HGF were added, followed by stirring and mixing for 11 minutes, thereby obtaining the thermosensitive submucosal injection solution.

In this thermosensitive submucosal injection, the chitosan concentration was 0.7% (w/v, g/mL), polyvinylpyrrolidone concentration was 8% (w/v, g/mL), disodium β-glycerophosphate concentration was 7% (w/v, g/mL), calcium ion concentration was 0.03% (w/v, g/mL), methylene blue concentration was 1% (v/v), epinephrine concentration was 0.009% (v/v), and each growth factor concentration was 50 ng/mL.

Additionally, two control group solutions were prepared by replacing the chitosan solution (pH 6.1) with conventional low-pH chitosan solution (pH 5.4) and higher-pH chitosan solution (pH 6.2), respectively, while keeping all other conditions unchanged. 0.5 mL aliquots of the experimental group and the two control groups were tested for gelation time in a 37° C. water bath. The experiments showed: the pH 6.1 chitosan solution group achieved gelation within 5 minutes; the pH 6.2 chitosan solution group achieved gelation within 3 minutes; and the low-pH chitosan solution (pH 5.4) failed to form a gel within 1 hour. Additionally, a third control group was prepared by replacing the chitosan solution (pH 6.1) with a higher-pH chitosan solution (pH 6.3) while keeping all other conditions unchanged. However, during the experiment, chitosan precipitation was observed under pH 6.3 conditions, and a clear chitosan solution could not be obtained, as this pH reached the isoelectric point of chitosan. These experiments demonstrated that the thermosensitive injection based on high-pH chitosan solutions (pH 5.5-6.2) proposed in the present disclosure enables stable preparation and rapid gelation.

### Example 2

Under ice bath and stirring conditions (7,000 rpm), a polyvinylpyrrolidone solution (molecular weight: 60,000 Da) was first added to a chitosan solution (degree of deacetylation: 96%, molecular weight: 140,000 Da, pH 6.18), followed by stirring and mixing for 32 minutes. Subsequently, a disodium β-glycerophosphate solution was dropwise added to the above mixture, followed by stirring and mixing for 12 minutes. Calcium gluconate solution was then added, followed by stirring and mixing for 14 minutes. Next, the medical color indicator indigo carmine was added, followed by stirring and mixing for 12 minutes. The hemostatic drug epinephrine was subsequently added, followed by stirring and mixing for 13 minutes. Finally, growth factors EGF, PDGF, and TGF-β were added, followed by stirring and mixing for 16 minutes, thereby obtaining the thermosensitive submucosal injection solution.

In this thermosensitive submucosal injection, the chitosan concentration was 0.6% (w/v, g/mL), polyvinylpyrrolidone concentration was 5% (w/v, g/mL), disodium β-glycerophosphate concentration was 5% (w/v, g/mL), calcium ion concentration was 0.04% (w/v, g/mL), indigo carmine concentration was 1% (v/v), epinephrine concentration was 0.013% (v/v), and each growth factor concentration was 80 ng/mL.

Additionally, a control group solution was prepared by replacing the chitosan solution (pH 6.18) with a slightly lower-pH chitosan solution (pH 5.8) while keeping all other conditions unchanged. 0.5 mL aliquots of both solutions were tested for gelation time in a 37° C. water bath. The experiments showed: the pH 6.18 chitosan solution group achieved gelation within 3 minutes; and the pH 5.8 chitosan solution group achieved gelation within 7 minutes. This experiment demonstrated that the injection solution prepared with higher-pH chitosan solutions achieves faster gelation under body temperature.

### Example 3

Under ice bath and stirring conditions (8,000 rpm), a polyvinylpyrrolidone solution (molecular weight: 70,000 Da) was first added to a chitosan solution (degree of deacetylation: 95%, molecular weight: 220,000 Da, pH 6.0), followed by stirring and mixing for 40 minutes. Subsequently, a disodium β-glycerophosphate solution was dropwise added to the above mixture, followed by stirring and mixing for 13 minutes. Calcium heparin solution was then added, followed by stirring and mixing for 14 minutes. Next, the medical color indicator methylene blue was added, followed by stirring and mixing for 18 minutes. The hemostatic drug epinephrine was subsequently added, followed by stirring and mixing for 12 minutes. Finally, growth factors bFGF and EGF were added, followed by stirring and mixing for 16 minutes, thereby obtaining the thermosensitive submucosal injection solution.

In this thermosensitive submucosal injection, the chitosan concentration was 0.55% (w/v, g/mL), polyvinylpyrrolidone concentration was 2% (w/v, g/mL), disodium β-glycerophosphate concentration was 7.5% (w/v, g/mL), calcium ion concentration was 0.02% (w/v, g/mL), methylene blue concentration was 1% (v/v), epinephrine concentration was 0.015% (v/v), and each growth factor concentration was 40 ng/mL.

Additionally, four control groups were prepared: (1) replacing the polyvinylpyrrolidone solution with water in the aforementioned steps; (2) polyvinylpyrrolidone concentration in the injection being 1.9% (w/v, g/mL); (3) polyvinylpyrrolidone concentration in the injection being 10% (w/v, g/mL); and (4) polyvinylpyrrolidone concentration in the injection being 11% (w/v, g/mL). The experiments showed that control groups (1) and (2) exhibited white turbidity, particularly most pronounced in the polyvinylpyrrolidone-free group, while the experimental group (containing 2% (w/v, g/mL) polyvinylpyrrolidone), control group (3), and control group (4) remained clear. Further gelation experiments in a 37° C. water bath revealed: control group (4) failed to form a gel within 1 hour; and the experimental group and control group (3) achieved gelation within 4 minutes and 8 minutes, respectively. These experiments demonstrated that polyvinylpyrrolidone at 2-10% (w/v, g/mL) forms an interpenetrating network with chitosan molecules through hydrogen bonding interactions. This partially reduces the strong electrostatic attraction between chitosan molecules, significantly improves injection stability, and enables rapid gelation at 37° C. When polyvinylpyrrolidone concentration is below 2% (w/v, g/mL), its hydrogen bonding interaction with chitosan molecules becomes extremely weak, resulting in poor injection stability and visible precipitation. Conversely, concentrations above 10% (w/v, g/mL) resulted in insufficient electrostatic attraction between chitosan molecules, preventing gel formation even after prolonged exposure to 37° C., rendering the solution unusable. Therefore, these experiments confirmed that the optimal final concentration range of polyvinylpyrrolidone in the present disclosure is 2 to 10% (w/v, g/mL).

### Example 4

Under ice bath and stirring conditions (1,500 rpm), a polyvinylpyrrolidone solution (molecular weight: 40,000 Da) was first added to a chitosan solution (degree of deacetylation: 99%, molecular weight: 25,000 Da, pH 6.08), followed by stirring and mixing for 22 minutes. Subsequently, a disodium β-glycerophosphate solution was dropwise added to the above mixture, followed by stirring and mixing for 19 minutes. Calcium gluconate solution was then added, followed by stirring and mixing for 12 minutes. Next, the medical color indicator indigo carmine was added, followed by stirring and mixing for 11 minutes. The hemostatic drug epinephrine was subsequently added, followed by stirring and mixing for 18 minutes. Finally, growth factors VEGF, EGF, and PDGF were added, followed by stirring and mixing for 20 minutes, thereby obtaining the thermosensitive submucosal injection solution.

In this thermosensitive submucosal injection, the chitosan concentration was 0.75% (w/v, g/mL), polyvinylpyrrolidone concentration was 7% (w/v, g/mL), disodium β-glycerophosphate concentration was 8% (w/v, g/mL), calcium ion concentration was 0.045% (w/v, g/mL), indigo carmine concentration was 1% (v/v), epinephrine concentration was 0.010% (v/v), and each growth factor concentration was 65 ng/mL. Additionally, two control groups were prepared: (1) polyvinylpyrrolidone concentration was adjusted to 3% (w/v, g/mL) with all other conditions unchanged; (2) calcium ion concentration was adjusted to 0.015% (w/v, g/mL) with all other conditions unchanged.

Bama minipigs were anesthetized, and the experimental group and two control group injections were locally injected into the esophageal submucosal layer via an endoscopic injection needle. Each injection site received 1 mL at an injection rate of 8 mL/min. The height of mucosal elevation and subsequent height changes over time were monitored using endoscopic ultrasound. The experiments showed: initial mucosal elevation heights were similar across all groups (approximately 10 mm); and at 40 minutes, the experimental group maintained a height of 7 mm, while control group (1) reduced to 4 mm and control group (2) reduced to 5 mm. This experiment demonstrated that increasing polyvinylpyrrolidone or calcium ion concentrations significantly slows the collapse of elevated mucosa, thereby facilitating ESD operations.

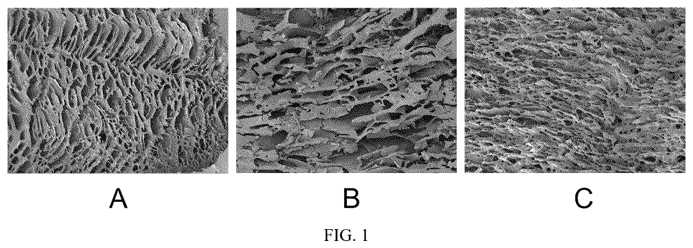

Additionally, to clarify the mechanisms by which polyvinylpyrrolidone and calcium ion concentrations delay mucosal layer collapse, scanning electron microscopy (SEM) analysis and gel strength measurements were further conducted on the gels from the experimental group and the two control groups described above. FIG. 1 shows cross-sectional SEM morphologies of the three groups: inset A: experimental group (polyvinylpyrrolidone and calcium ion concentrations: 7% (w/v, g/mL) and 0.045% (w/v, g/mL), respectively); inset B: control group (1) (polyvinylpyrrolidone and calcium ion concentrations: 3% (w/v, g/mL) and 0.045% (w/v, g/mL), respectively); and inset C: control group (2) (polyvinylpyrrolidone and calcium ion concentrations: 7% (w/v, g/mL) and 0.015% (w/v, g/mL), respectively). Inset A revealed that the experimental group, with higher concentrations of both polyvinylpyrrolidone and calcium ions, exhibited a relatively compact gel structure with highly ordered organization. Inset B showed that the structural compactness significantly decreased when polyvinylpyrrolidone concentration was reduced (control group 1), though a certain degree of organizational order was retained. Inset C demonstrated that the structural ordering markedly declined when calcium ion concentration was lowered (control group 2), while relatively high compactness was maintained. The SEM morphological results indicated that increasing polyvinylpyrrolidone concentration enhanced gel structural compactness, while increasing calcium ion concentration improved structural ordering. Additionally, gel strength measurements revealed storage modulus values of 10.5 Pa for the experimental group, 5.2 Pa for control group (1), and 8.0 Pa for control group (2). These results demonstrated that reducing polyvinylpyrrolidone concentration significantly decreases gel strength, and lowering calcium ion concentration also reduces gel strength. Combining the SEM morphological analysis, gel strength measurements, and in vivo injection results confirms: reduced polyvinylpyrrolidone concentration decreases gel structural compactness, thereby ultimately leading to diminished mucosal elevation efficacy; reduced calcium ion concentration decreases gel structural ordering, thereby also reducing gel strength and ultimately diminishing mucosal elevation efficacy. The regulatory mechanisms of calcium ion concentration differ significantly from those of polyvinylpyrrolidone.

### Example 5

Under ice bath and stirring conditions (1,500 rpm), a polyvinylpyrrolidone solution (molecular weight: 40,000 Da) was first added to a chitosan solution (degree of deacetylation: 99%, molecular weight: 25,000 Da, pH 6.08), followed by stirring and mixing for 22 minutes. Subsequently, a disodium β-glycerophosphate solution was dropwise added to the above mixture, followed by stirring and mixing for 19 minutes. Calcium gluconate solution was then added, followed by stirring and mixing for 12 minutes. Next, the medical color indicator indigo carmine was added, followed by stirring and mixing for 11 minutes. The hemostatic drug epinephrine was subsequently added, followed by stirring and mixing for 18 minutes. Finally, growth factors VEGF, EGF, and PDGF were added, followed by stirring and mixing for 20 minutes, thereby obtaining the thermosensitive submucosal injection solution.

In this thermosensitive submucosal injection, the chitosan concentration was 0.75% (w/v, g/mL), polyvinylpyrrolidone concentration was 7% (w/v, g/mL), disodium β-glycerophosphate concentration was 8% (w/v, g/mL), calcium ion concentration was 0.03% (w/v, g/mL), indigo carmine concentration was 1% (v/v), epinephrine concentration was 0.01% (v/v), and each growth factor concentration was 65 ng/mL.

Additionally, five control groups were prepared: (1) calcium ion concentration adjusted to 0.0095% (w/v, g/mL), all other conditions unchanged; (2) calcium ion concentration adjusted to 0.01% (w/v, g/mL), all other conditions unchanged; (3) calcium ion concentration adjusted to 0.0105% (w/v, g/mL), all other conditions unchanged; (4) calcium ion concentration adjusted to 0.05% (w/v, g/mL), all other conditions unchanged; and (5) calcium ion concentration adjusted to 0.055% (w/v, g/mL), all other conditions unchanged. During preparation of low-temperature injection solutions, control group (5) exhibited turbidity due to chitosan flocculation caused by excessive calcium ion concentration, while other groups were successfully prepared and formed gels at 37° C. This confirms that the upper limit of calcium ion concentration is 0.05% (w/v, g/mL). Further, gel strength measurements on the experimental group and control groups (1)-(4) showed storage modulus values of 9.3 Pa, 7.2 Pa, 7.2 Pa, 7.7 Pa, and 12.6a, respectively. These results indicated that gel strength undergoes marked changes only when calcium ion concentration exceeds 0.01% (w/v, g/mL), thus establishing 0.01% (w/v, g/mL) as the minimum effective calcium ion concentration. Moreover, increasing calcium ion concentration was observed to enhance gel strength.

### Example 6

Under ice bath and stirring conditions (8,500 rpm), a polyvinylpyrrolidone solution (molecular weight: 120,000 Da) was first added to a chitosan solution (degree of deacetylation: 96%, molecular weight: 15,000 Da, pH 6.15), followed by stirring and mixing for 26 minutes. Subsequently, a disodium β-glycerophosphate solution was dropwise added to the above mixture, followed by stirring and mixing for 10 minutes. Calcium lactate solution was then added, followed by stirring and mixing for 12 minutes. Next, the medical color indicator methylene blue was added, followed by stirring and mixing for 12 minutes. The hemostatic drug epinephrine was subsequently added, followed by stirring and mixing for 13 minutes. Finally, growth factors bFGF and VEGF were added, followed by stirring and mixing for 15 minutes, thereby obtaining the thermosensitive submucosal injection solution.

In this thermosensitive submucosal injection, the chitosan concentration was 0.7% (w/v, g/mL), polyvinylpyrrolidone concentration was 4% (w/v, g/mL), disodium β-glycerophosphate concentration was 6.5% (w/v, g/mL), calcium ion concentration was 0.02% (w/v, g/mL), methylene blue concentration was 1% (v/v), epinephrine concentration was 0.02% (v/v), and each growth factor concentration was 30 ng/mL. Additionally, a control group injection without epinephrine was prepared by replacing epinephrine with water in the aforementioned steps.

Bama minipigs were anesthetized, and both the experimental injection (containing epinephrine) and the epinephrine-free control group injection were locally injected into the esophageal submucosal layer via an endoscopic injection needle. Each injection site received 1.2 mL at an injection rate of 13 mL/min. ESD procedures were then performed to resect the mucosal layer, with intraoperative and postoperative bleeding observed. The experiments showed: the epinephrine-containing experimental group exhibited no significant bleeding during or after surgery; and the epinephrine-free control group experienced intraoperative bleeding with obscured visual fields, prolonged surgical time, and postoperative bleeding. This experiment demonstrated that the injection containing epinephrine effectively reduces intraoperative and postoperative bleeding, thereby alleviating ESD complications.

### Example 7

Under ice bath and stirring conditions (6,500 rpm), a polyvinylpyrrolidone solution (molecular weight: 100,000 Da) was first added to a chitosan solution (degree of deacetylation: 95%, molecular weight: 200,000 Da, pH 6.05), followed by stirring and mixing for 35 minutes. Subsequently, a disodium β-glycerophosphate solution was dropwise added to the above mixture, followed by stirring and mixing for 16 minutes. Calcium chloride solution was then added, followed by stirring and mixing for 14 minutes. Next, the medical color indicator indigo carmine was added, followed by stirring and mixing for 11 minutes. The hemostatic drug epinephrine was subsequently added followed by stirring and mixing for 12 minutes. Finally, growth factors EGF, TGF-β, and HGF were added, followed by stirring and mixing for 14 minutes, thereby obtaining the thermosensitive submucosal injection solution.

In this thermosensitive submucosal injection, the chitosan concentration was 0.5% (w/v, g/mL), polyvinylpyrrolidone concentration was 4% (w/v, g/mL), disodium β-glycerophosphate concentration was 4% (w/v, g/mL), calcium ion concentration was 0.02% (w/v, g/mL), indigo carmine concentration was 1% (v/v), epinephrine concentration was 0.010% (v/v), and each growth factor concentration was 100 ng/mL. Additionally, a control group injection without growth factors was prepared by replacing growth factors with water in the aforementioned steps.

Bama minipigs were anesthetized, and both the experimental injection (containing growth factors) and the control group injection were locally injected into the esophageal submucosal layer via an endoscopic injection needle. Each injection site received 0.8 mL at an injection rate of 8 mL/min. ESD procedures were performed to resect the mucosal layer, with postoperative wound healing monitored over one month through periodic follow-up examinations. The experiments revealed that the growth factor-containing experimental group exhibited the earliest wound healing, demonstrating that the incorporation of growth factors accelerates wound recovery.

### Example 8

Under ice bath and stirring conditions (1,000 rpm), a polyvinylpyrrolidone solution (molecular weight: 300,000 Da) was first added to a chitosan solution (degree of deacetylation: 95%, molecular weight: 5,000 Da, pH 6.2), followed by stirring and mixing for 20 minutes. Subsequently, a disodium β-glycerophosphate solution was dropwise added to the above mixture, followed by stirring and mixing for 20 minutes. Calcium chloride solution was then added, followed by stirring and mixing for 10 minutes, thereby obtaining the thermosensitive submucosal injection solution. In this thermosensitive submucosal injection, the chitosan concentration was 1% (w/v, g/mL), polyvinylpyrrolidone concentration was 2% (w/v, g/mL), disodium β-glycerophosphate concentration was 10% (w/v, g/mL), and calcium ion concentration was 0.05% (w/v, g/mL).

Additionally, a selenium nanoparticle-loaded thermosensitive chitosan hydrogel solution was prepared as a control group. First, 3 g of chitosan powder (degree of deacetylation: 95%, molecular weight: 5,000 Da) was weighed and added to 120 mL of 1 wt % acetic acid solution. The mixture was stirred overnight until completely dissolved, autoclaved at 121° C. for 10 minutes, and stored at 4° C. to obtain a 2.5% (w/v, g/mL) chitosan solution, with measured pH 5.2. This pH 5.2 chitosan solution was placed under ice bath stirring (500 rpm). A selenium nanoparticle solution (6.25 μg/mL or 100 μg/mL) was slowly added and stirred for 30 minutes. Subsequently, a 70% (w/v, g/mL) disodium β-glycerophosphate solution was dropwise added using a peristaltic pump at a rate of 23 rpm, followed by stirring for 30 minutes, yielding two control injections. The two control injections contained chitosan at concentration of 1.67% (w/v, g/mL), disodium β-glycerophosphate at concentration of 9.33% (w/v, g/mL), and selenium nanoparticles at concentrations of 1.25 μg/mL and 20 μg/mL, respectively.

The experimental group and both control groups contained no medical color indicators, epinephrine, or growth factors. 0.5 mL aliquots of all three groups were tested for gelation time in a 37° C. water bath. The results showed: the experimental group achieved gelation within 2 minutes; the 1.25 μg/mL and 20 μg/mL selenium nanoparticle control groups achieved gelation within 12 minutes and 10 minutes, respectively. This indicates the experimental group exhibited significantly shorter gelation times compared to the control groups. Additionally, viscosity measurements revealed: the experimental group had a viscosity of 55 cp; the two control groups showed viscosities of 118 cp and 143 cp, respectively. This demonstrates the experimental group had significantly lower viscosity than both control groups. Furthermore, Bama minipigs were anesthetized, and the three injection solutions were locally injected into the esophageal submucosal layer via an endoscopic injection needle. The experimental group allowed smooth injection of 1 mL per site at 10 mL/min, while both control groups exhibited excessively high viscosity, creating extreme resistance that prevented injection through the endoscopic needle at any speed. These in vitro and in vivo experiments confirm that the injection solution of the present disclosure outperforms selenium nanoparticle-loaded thermosensitive chitosan hydrogel injection solutions.

### Example 9

Under ice bath and stirring conditions (1,000 rpm), a polyvinylpyrrolidone solution (molecular weight: 300,000 Da) was first added to a chitosan solution (degree of deacetylation: 95%, molecular weight: 5,000 Da, pH 6.2), followed by stirring and mixing for 20 minutes. Subsequently, a disodium β-glycerophosphate solution was dropwise added to the above mixture, followed by stirring and mixing for 20 minutes. Calcium chloride solution was then added, followed by stirring and mixing for 10 minutes. Next, the medical color indicator methylene blue was added, followed by stirring and mixing for 10 minutes. The hemostatic drug epinephrine was subsequently added, followed by stirring and mixing for 20 minutes. Finally, growth factors bFGF, VEGF, and EGF were added, followed by stirring and mixing for 10 minutes, thereby obtaining the thermosensitive submucosal injection solution.

In this thermosensitive submucosal injection, the chitosan concentration was 1% (w/v, g/mL), polyvinylpyrrolidone concentration was 2% (w/v, g/mL), disodium β-glycerophosphate concentration was 10% (w/v, g/mL), calcium ion concentration was 0.05% (w/v, g/mL), methylene blue concentration was 1% (v/v), epinephrine concentration was 0.005% (v/v), and each growth factor concentration was 20 ng/mL.

Bama minipigs were anesthetized, and the aforementioned injection solution along with normal saline containing methylene blue (control group) were locally injected into the esophageal submucosal layer via an endoscopic injection needle. Each injection site received 0.5 mL at an injection rate of 5 mL/min. Endoscopic ultrasound was used to measure the initial mucosal elevation height and subsequent height changes over time. ESD procedures were then performed to resect the mucosal layer, with surgical duration recorded and intraoperative bleeding/perforation observed. Postoperative wound healing was monitored over one month through periodic follow-up examinations.

The experiments revealed: The thermosensitive submucosal injection group and normal saline group showed mucosal elevation heights of 6 mm and 4 mm immediately post-injection, respectively, with the thermosensitive injection group exhibiting significantly higher elevation. Additionally, the normal saline group exhibited complete collapse of mucosal elevation within 5 minutes post-injection, whereas the thermosensitive submucosal injection group maintained a height of 4 mm even at 30 minutes post-injection. These results demonstrate that the thermosensitive submucosal injection effectively sustained mucosal elevation. Additionally, immediate mucosal layer resection post-injection revealed that the normal saline group required repeated saline injections, whereas the thermosensitive submucosal injection group had already formed a gel, eliminating the need for reinjection. Additionally, intraoperative observations showed: the normal saline group exhibited bleeding, obscured visual fields, and perforation; and the thermosensitive submucosal injection group displayed no significant bleeding, maintained clear visual fields, demonstrated markedly shorter resection time compared to the normal saline group, and showed no perforation. At 1 month postoperatively, wounds in the thermosensitive submucosal injection group had fully healed, whereas the normal saline group remained incompletely healed. This indicates that the thermosensitive submucosal injection formed a protective barrier over the wound postoperatively, accelerating healing through synergistic effects of chitosan and growth factors.

### Example 10

Under ice bath and stirring conditions (10,000 rpm), a polyvinylpyrrolidone solution (molecular weight: 20,000 Da) was first added to a chitosan solution (degree of deacetylation: 99%, molecular weight: 300,000 Da, pH 5.5), followed by stirring and mixing for 40 minutes. Subsequently, a disodium β-glycerophosphate solution was dropwise added to the above mixture, followed by stirring and mixing for 10 minutes. Calcium lactate solution was then added, followed by stirring and mixing for 20 minutes. Next, the medical color indicator methylene blue was added, followed by stirring and mixing for 20 minutes. The hemostatic drug epinephrine was subsequently added, followed by stirring and mixing for 10 minutes. Finally, growth factors PDGF, TGF-β, and HGF were added, followed by stirring and mixing for 20 minutes, thereby obtaining the thermosensitive submucosal injection solution.

In this thermosensitive submucosal injection, the chitosan concentration was 0.3% (w/v, g/mL), polyvinylpyrrolidone concentration was 10% (w/v, g/mL), disodium β-glycerophosphate concentration was 2% (w/v, g/mL), calcium ion concentration was 0.010% (w/v, g/mL), methylene blue concentration was 10% (v/v), epinephrine concentration was 0.02% (v/v), and each growth factor concentration was 100 ng/mL.

Bama minipigs were anesthetized, and the aforementioned injection solution along with normal saline containing methylene blue (control group) were locally injected into the esophageal submucosal layer via an endoscopic injection needle. Each injection site received 1.5 mL at an injection rate of 15 mL/min. Endoscopic ultrasound was used to measure the initial mucosal elevation height and subsequent height changes over time. ESD procedures were then performed to resect the mucosal layer, with surgical duration recorded and intraoperative bleeding/perforation observed. Postoperative wound healing was monitored over one month through periodic follow-up examinations.

The experiments revealed: The thermosensitive submucosal injection group and normal saline group showed mucosal elevation heights of 14 mm and 9 mm immediately post-injection, respectively, with the thermosensitive injection group exhibiting significantly higher elevation. Additionally, the normal saline group exhibited complete collapse of mucosal elevation within 5 minutes post-injection, whereas the thermosensitive submucosal injection group maintained a height of 10 mm even at 30 minutes post-injection. These results demonstrate that the thermosensitive submucosal injection effectively sustained mucosal elevation. Additionally, immediate mucosal layer resection post-injection revealed that the normal saline group required repeated saline injections, whereas the thermosensitive submucosal injection group had already formed a gel, eliminating the need for reinjection. Additionally, intraoperative observations showed: the normal saline group exhibited bleeding and obscured visual fields; and the thermosensitive submucosal injection group displayed no significant bleeding, maintained clear visual fields, demonstrated markedly shorter resection time compared to the normal saline group. At 1 month postoperatively, wounds in the thermosensitive submucosal injection group had fully healed, whereas the normal saline group remained incompletely healed. This indicates that the thermosensitive submucosal injection formed a protective barrier over the wound postoperatively, accelerating healing through synergistic effects of chitosan and growth factors.

### Example 11

Under ice bath and stirring conditions (5,500 rpm), a polyvinylpyrrolidone solution (molecular weight: 160,000 Da) was first added to a chitosan solution (degree of deacetylation: 97%, molecular weight: 152,500 Da, pH 5.85), followed by stirring and mixing for 30 minutes. Subsequently, a disodium β-glycerophosphate solution was dropwise added to the above mixture, followed by stirring and mixing for 15 minutes. Calcium chloride solution was then added, followed by stirring and mixing for 15 minutes. Next, the medical color indicator indigo carmine was added, followed by stirring and mixing for 15 minutes. The hemostatic drug epinephrine was subsequently added, followed by stirring and mixing for 15 minutes. Finally, growth factors VEGF, EGF, TGF-β, and HGF were added, followed by stirring and mixing for 15 minutes, thereby obtaining the thermosensitive submucosal injection solution.

In this thermosensitive submucosal injection, the chitosan concentration was 0.65% (w/v, g/mL), polyvinylpyrrolidone concentration was 6% (w/v, g/mL), disodium β-glycerophosphate concentration was 6% (w/v, g/mL), calcium ion concentration was 0.03% (w/v, g/mL), indigo carmine concentration was 1% (v/v), epinephrine concentration was 0.0125% (v/v), and each growth factor concentration was 60 ng/mL.

Bama minipigs were anesthetized, and the aforementioned injection solution along with normal saline containing methylene blue (control group) were locally injected into the esophageal submucosal layer via an endoscopic injection needle. Each injection site received 1 mL at an injection rate of 10 mL/min. Endoscopic ultrasound was used to measure the initial mucosal elevation height and subsequent height changes over time. ESD procedures were then performed to resect the mucosal layer, with surgical duration recorded and intraoperative bleeding/perforation observed. Postoperative wound healing was monitored over one month through periodic follow-up examinations.

The experiments revealed: The thermosensitive submucosal injection group and normal saline group showed mucosal elevation heights of 10 mm and 7.5 mm immediately post-injection, respectively, with the thermosensitive injection group exhibiting significantly higher elevation. Additionally, the normal saline group exhibited complete collapse of mucosal elevation within 5 minutes post-injection, whereas the thermosensitive submucosal injection group maintained a height of 7 mm even at 30 minutes post-injection. These results demonstrate that the thermosensitive submucosal injection effectively sustained mucosal elevation. Additionally, immediate mucosal layer resection post-injection revealed that the normal saline group required repeated saline injections, whereas the thermosensitive submucosal injection group had already formed a gel, eliminating the need for reinjection. Additionally, intraoperative observations showed: the normal saline group exhibited bleeding, obscured visual fields; and the thermosensitive submucosal injection group displayed no significant bleeding, maintained clear visual fields, demonstrated markedly shorter resection time compared to the normal saline group. At 1 month postoperatively, wounds in the thermosensitive submucosal injection group had fully healed, whereas the normal saline group remained incompletely healed. This indicates that the thermosensitive submucosal injection formed a protective barrier over the wound postoperatively, accelerating healing through synergistic effects of chitosan and growth factors.

The above descriptions merely represent preferred embodiments of the present disclosure and shall not be construed as limiting the present disclosure. Any modifications, equivalent replacements, improvements, and the like made within the spirit and principles of the present disclosure shall be encompassed within the protection scope of the present disclosure.

1. A method for preparing a thermosensitive submucosal injection for endoscopic submucosal dissection, wherein the method comprises following steps:
S1. adding a polyvinylpyrrolidone solution to a chitosan solution with a high-pH value under ice bath and stirring conditions, followed by stirring and mixing uniformly to prepare a mixed solution A;
S2. adding a disodium β-glycerophosphate solution dropwise to the mixed solution A of S1, followed by stirring and mixing uniformly to prepare a mixed solution B;
S3. adding a calcium ion solution to the mixed solution B prepared in S2, followed by stirring and mixing uniformly to prepare a mixed solution C;
S4. adding a medical color indicator to the mixed solution C prepared in S3, followed by stirring and mixing uniformly to prepare a mixed solution D;
S5. adding a hemostatic drug epinephrine to the mixed solution D prepared in S4, followed by stirring and mixing uniformly to prepare a mixed solution E;
S6. adding a growth factor to the mixed solution E prepared in S5, followed by stirring and mixing uniformly to obtain the thermosensitive submucosal injection solution.

2. The method for preparing a thermosensitive submucosal injection for endoscopic submucosal dissection according to claim 1, wherein the high-pH value in step S1 is in a range of 5.5 to 6.2.

3. The method for preparing a thermosensitive submucosal injection for endoscopic submucosal dissection according to claim 1, wherein, in step S1, the chitosan has a degree of deacetylation of 95% or more and a molecular weight of 5,000-300,000 Da; the polyvinylpyrrolidone has a molecular weight of 20,000-300,000 Da.

4. The method for preparing a thermosensitive submucosal injection for endoscopic submucosal dissection according to claim 1, wherein specific conditions of the stirring in steps S1 to S6 are: a stirring and mixing time of 20 to 40 minutes in step S1; a stirring and mixing time of 10 to 20 minutes in steps S2 to S6; and a stirring speed of 1,000 to 10,000 rpm in steps S1 to S6.

5. The method for preparing a thermosensitive submucosal injection for endoscopic submucosal dissection according to claim 1, wherein the calcium ion solution in step S3 comprises one or more of calcium chloride, calcium heparin, calcium lactate, and calcium gluconate.

6. The method for preparing a thermosensitive submucosal injection for endoscopic submucosal dissection according to claim 1, wherein the medical color indicator in step S4 comprises any one of methylene blue and indigo carmine, or a combination thereof.

7. The method for preparing a thermosensitive submucosal injection for endoscopic submucosal dissection according to claim 1, wherein the growth factor in step S6 comprises any one of bFGF, VEGF, EGF, PDGF, TGF-β, and HGF, or a combination thereof.

8. The method for preparing a thermosensitive submucosal injection for endoscopic submucosal dissection according to claim 1, wherein in the thermosensitive submucosal injection solution prepared in step S6: the chitosan has a concentration of 0.3 to 1% (w/v, g/mL); the polyvinylpyrrolidone has a concentration of 2 to 10% (w/v, g/mL); the disodium β-glycerophosphate has a concentration of 2 to 10% (w/v, g/mL); the calcium ions have a concentration of 0.01 to 0.05% (w/v, g/mL); the medical color indicator has a concentration of 1% (v/v); the epinephrine has a concentration of 0.005 to 0.02% (v/v); and each growth factor has a concentration of 20 to 100 ng/mL.
